(19) United States
(12) Patent Application Publication
(10) Pub. No.: US 2023/0043862 A1
(43) Pub. Date: Feb. 9, 2023

(54) ALGORITHMS FOR SELECTING ATHLETIC AND RECOVERY EQUIPMENT,DEVICES, AND SOLUTIONS BASED ON MUSCLE DATA, AND ASSOCIATED SYSTEMS AND METHODS

(71) Applicant: Strive Tech Inc., Bothell, WA (US)

(72) Inventors: Nikola Mrvaljevic, Bothell, WA (US); Michael Mikhaylov, Bothell, WA (US)

(21) Appl. No.: 17/883,459

(22) Filed: Aug. 8, 2022

Related U.S. Application Data
(60) Provisional application No. 63/230,635, filed on Aug. 6, 2021.

(51) Int. Cl. A61B 5/397; A61B 5/11; A61B 5/00; G16H 20/30

(52) U.S. Cl. CPC A61B 5/397; A61B 5/1118; A61B 5/6801; G16H 20/30

(57) ABSTRACT

Systems and methods for providing algorithmic equipment and/or accessory recommendations are disclosed herein. In one embodiment, a method providing an equipment or accessory recommendation to an athlete includes: monitoring a first amplitude of a first muscle of the athlete by a first wearable muscle response sensor carried by the athlete; monitoring a second amplitude of a second muscle of the athlete by a second wearable muscle response sensor carried by the athlete; determining a difference between the first amplitude and the second amplitude; comparing the difference to a predetermined amplitude threshold; and based on the comparing, providing an equipment or accessory recommendation to the athlete.

### CROSS-REFERENCE TO RELATED APPLICATIONS

This application claims the benefit of provisional patent application number U.S. 63/230,635 filed Aug. 6, 2021, the contents of which are incorporated herein by reference.

### BACKGROUND

It is well known that the athletes, whether professional or otherwise, are subject to improper posturing, inefficient running, uneven use of muscles and other deficiencies. In some cases, these issues may lead to too early fatigue or injury. At least some of these issues can be improved when the athlete uses well suited equipment, e.g., proper shoes, athletic dress suitable for the purpose, set of weights, etc. (collectively, athletic equipment) that is appropriate for the athlete and for particular activity, etc. In addition, appropriate treatment or service equipment and/or supplements may improve athletic performance and reduce recovery time.

However, identifying well suited equipment, accessories, services, and/or supplements may require assistance of professionals. In some examples, even a professional may take several iterations to arrive at a suitable set of recommendations that takes into account particular needs or sometimes particular idiosyncrasies of an athlete. Therefore, assistance of such professionals is both time consuming and expensive. Accordingly, there remains a need for systems and methods to accurately generate recommendations for proper athletic equipment and/or accessories for the athletes.

### BRIEF DESCRIPTION OF THE DRAWINGS

The foregoing aspects and many of the attendant advantages will become more readily appreciated with reference to the following detailed description, when taken in conjunction with the accompanying drawings, where:

FIG. 1 is a schematic diagram illustrating an analytics system configured, in accordance with various embodiments.

FIG. 2 is a schematic diagram illustrating components of an example analytics system in further detail, in accordance with various embodiments.

FIG. 3 is block diagram illustrating components that can be incorporated into a computing device, in accordance with various embodiments.

FIG. 4A and FIG. 4B are diagrams showing a measurement system in accordance with various embodiments, in accordance with various embodiments.

FIG. 5 illustrates example muscle amplitude measurements for right quad (RQ) and left quad (LQ), in accordance with various embodiments.

FIG. 6 illustrates example muscle amplitude measurements for right hamstring (RH, solid line) and left hamstring (LH, dash line), in accordance with various embodiments.

FIG. 7 illustrates example muscle amplitude measurements for left hamstring (LH) and left glute (LG), in accordance with various embodiments.

FIG. 8 illustrates example acceleration and activity state measurements, in accordance with various embodiments.

FIG. 9 is an example flow for algorithmic equipment recommendations, in accordance with various embodiments.

### DETAILED DESCRIPTION

Embodiments are directed to generating individualized recommendations for an athlete's equipment, treatment equipment and/or accessories, supplements, and/or services. In the context of this application, the term athlete encompasses professional and amateur athletes, as well as hobbyists, people who exercise, on either a regular or an irregular basis, and others who engage in sports or exercise. All such categories of people (professional, amateur, consumers, etc.) are referred to as “athletes” in this application for simplicity and brevity.

In some embodiments, the athlete's equipment and/or accessories, such as a uniform or other exercise clothing, may be equipped with suitable sensors and/or data acquisition controllers that collect and interpret muscle activity data (e.g., muscle amplitude and frequency, heart rate, etc.). Such sensors may measure electrical impulses of the muscles representing muscle activity data. Collected data may be algorithmically processed to indicate muscle amplitude and/or frequency for one or more muscle groups of the user. In some embodiments, the algorithmic processing may include artificial intelligence and/or machine learning models.

In some embodiments, individualized recommendations for athlete's equipment and/or accessories are based on measured differences between particular groups of muscles and motion of the athlete during exercise or physical therapy. For example, muscle and motion data can be measured. Based on, for example, running preference, inventive systems and method may focus on recommending proper shoes or foot orthotics. Such recommendations may be made based on a difference in the muscle output of different groups of muscles, for example, Quads and Glute groups. Another non-limiting example of a basis for equipment recommendation is ankle movement. For example, recommendation for proper shoes may be made by identifying incorrect running, walking, or posturing by an athlete. When properly selected, recommended shoes and/or other athletic equipment or accessories may improve athlete's running, walking, gait, posturing, etc.

In some embodiments, accelerometer and/or other inertial measurement units (IMUs) are added to shoes in order to collect both the muscle data and the foot movement data. Foot movement may be important in estimating for example, whether athletes under/over-pronate.

Motion information may inform the determination of fatigue or injury and may be applied to reduce the likelihood of future injury, to improve performance, or the like, through recommendations for new or different equipment for training, recovery, or competition, as well as services and/or supplements. For example, if the right hamstring is not recording a proper output at a low level of motion, a system may recommend a sleeve or hamstring tape to support the right hamstring. By contrast, at higher levels of motion, which may include multiple graduations or levels, the system may suggest replacement and/or different footwear. Collectively, such recommendations for equipment or garments are herein referred to as an equipment recommendation. In many embodiments, such an early and rapid recommendation may protect the athlete from further deterioration due to fatigue or injury, may promote improvement at the relevant activity, all while being significantly more cost effective than conventional methods where the athlete is evaluated by an expert or otherwise finds a well-suited piece of equipment by trial and error.

The forthcoming description focuses on selection and recommendation of athletic equipment and/or accessories for use during training and/or competition. Selection and recommendation may also include, but is not limited to, treatment and/or recovery equipment, supplements, and/or services. For example, treatment equipment may include, but is not limited to massagers, massage devices, muscle/tissue manipulators, muscle percussion devices, or heating and/or cooling devices (e.g., straps, single-use items, etc.). In some embodiments, treatment equipment may also include, but is similarly not limited to, air compression devices, Transcutaneous Electrical Nerve Stimulation (TENS) machines, electrical muscle stimulators (EMS devices), electronic stimulators (e-stim), or cryotherapy devices. In some embodiments, supplements may include, but are not limited to, electrolyte supplements and/or nutritional supplements (e.g., protein, amino acid, vitamin, mineral, etc.). In some embodiments, services may include, but are not limited to massages, nutritional services, TENS treatments, EMS treatments, e-stim treatments, thermal treatments, or cryotherapy treatments.

System Overview

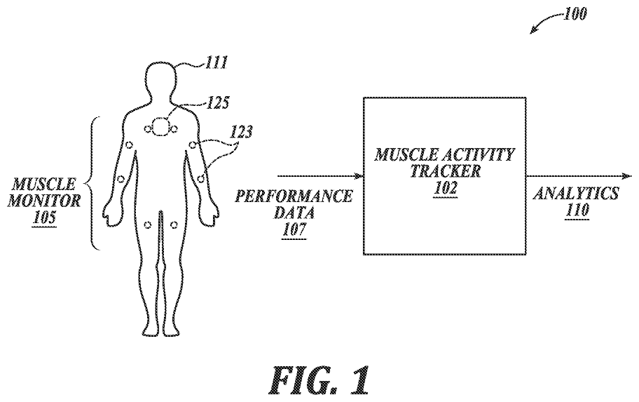

FIG. 1 is a schematic diagram illustrating an example analytics system 100 configured in accordance with an embodiment of the present technology. The system 100 includes a muscle activity tracker sub-system 102 (“muscle activity tracker 102”) and a muscle monitoring sub-system 105 (“muscle monitor 105”) that is worn by a user, such as an athlete or a user 111. The muscle monitor 105 may include an on-board controller 125 (“controller 125”) and sensors 123 that can be integrated into the athlete's clothing (not shown), such as the athlete's shirt, pants, shoes, etc. The athlete's clothing and the integrated controller 125 and sensors 123 may be collectively referred to as “smart compression clothing.” In operation, the controller 125 is configured to produce real-time or near real-time performance data (“real-time data”) 107 during an exercise, live game, practice session, or conditioning. Analytics 110 may include muscle response (MR) data, like frequency and amplitude activity for different groups of muscles, as well as motion information, as may be collected by a wearable accelerometer borne by the athlete. In different embodiments, analytics 110 may include data related to orientation state (OS) of the user, acceleration of the user, activity state (AS) of the user, etc. The analytics 110 may be produced over an evaluation period of a certain duration (e.g., 1 hour, 30 minutes, 15 minutes, 5 minutes, etc.). As described below, the system 100 can use the analytics 110 to produce indications, warnings, and alarms that alert the user or the trainer when an athlete is fatigued or injured. The system 100 can also produce indications of whether athlete's posturing, running, walking, etc. is appropriate for a given activity, and/or whether the athlete is using proper equipment and/or accessories (e.g., shoes, uniform, exercise weights, insoles, joint sleeves, muscle tape, electronic peripherals such as heart monitors, etc.).

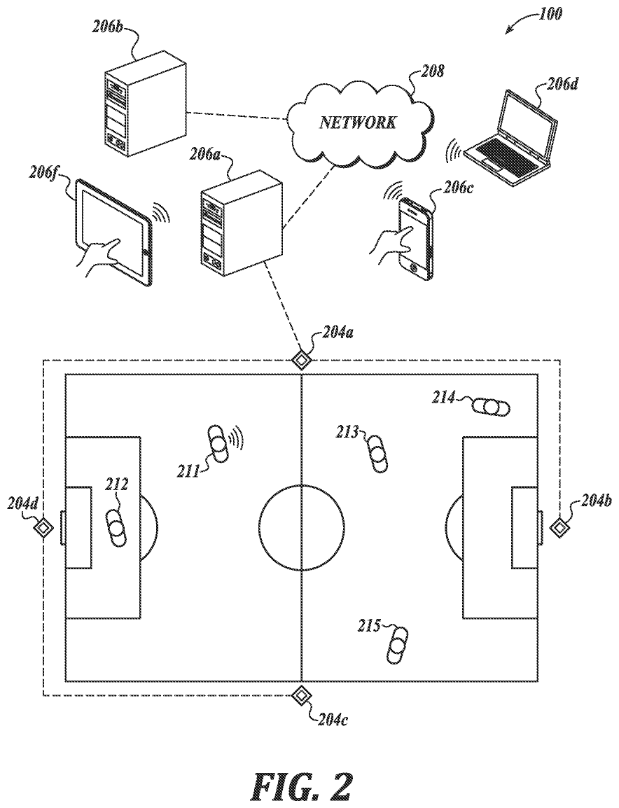

FIG. 2 is a schematic diagram illustrating components of an example analytics system 100 in further detail, in accordance with various embodiments. The system 100 illustrates interactions with multiple athletes, however, in other embodiments, the system may be focused on a single athlete. Furthermore, in different embodiments, the system 100 may include a subset of the illustrated components or additional components to those that are illustrated.

The muscle monitor 105 shown in FIG. 1 may be configured to communicate with one or more computing devices 206 via a plurality of gateway devices 204 positioned along monitoring region 227, such as a soccer-field, an athletic arena, gym, etc. The computing devices 206 are connected to one another via a network 208. The computing devices 206 are configured to receive, view, evaluate, store, and/or otherwise interact with data associated with the analytics 110 (FIG. 1). For example, intermediary or back-end server devices 206a and 206b can exchange and process communications over the network 208, store a central copy of data, globally update content, etc. Examples of well-known computing devices, systems, environments, and/or configurations that may be suitable for use with the technology include, but are not limited to, personal computers, server computers, handheld or laptop devices, cellular telephones, tablet devices, multiprocessor systems, microprocessor-based systems, set-top boxes, programmable consumer electronics, network PCs, minicomputers, mainframe computers, databases, distributed computing environments that include any of the above systems or devices, or the like.

One or more computing devices 206 can be configured to individually or collectively carry out the functions of the performance tracker 102 (FIG. 1) for producing the analytics 110. In various embodiments, the various computing devices 206 can process real-time data produced by one or more athletes 211-215 that are monitored in the monitoring region 227 of the gateways 204. As described below, the gateways 204 are configured to forward the real-time data 107 (FIG. 1) to the upstream computing devices 206 for processing.

Computing Devices

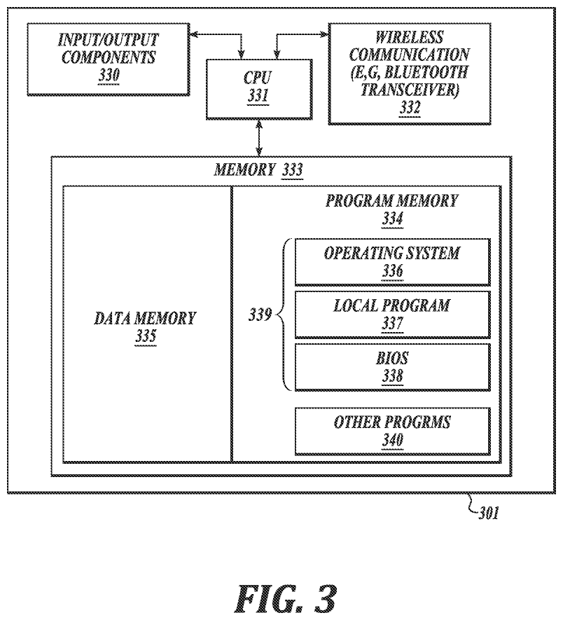

FIG. 3 is block diagram illustrating components that can be incorporated into a computing device 301, such as one of the computing devices 206 (FIG. 3), the gateways 204 (FIG. 3), and the muscle monitor 105 (1A). The computing device 301 includes input and output components 330. Input components can be configured to provide input to a processor such as CPU 331, notifying it of actions. The actions are typically mediated by a hardware controller that communicates according to one or more communication protocols. The input components 330 can include, for example, a mouse, a keyboard, a touchscreen, an infrared sensor, a touchpad, a pointer device, a camera- or image-based input device, a pointer, and/or a microphone.

The CPU 331 can be a single processing unit or multiple processing units in a device or distributed across multiple devices. The CPU 331 can be coupled to other hardware components via, e.g., a bus, such as a PCI bus or SCSI bus. Other hardware components can include communication components 332, such as a wireless transceiver (e.g., a WiFi or Bluetooth transceiver) and/or a network card. Such communication components 332 can enable communication over wired or wireless (e.g., point-to point) connections with other devices. A network card can enable the computing device 301 to communicate over the network 208 (FIG. 3) using, e.g., TCP/IP protocols. Additional hardware components may include other input/output components, including a display, a video card, audio card, USB, firewire, or other external components or devices, such as a camera, printer, thumb drive, disk drive, Blu-Ray device, and/or speakers.

The CPU 331 can have access to a memory 333. The memory 333 includes volatile and non-volatile components which may be writable or read-only. For example, the memory can comprise CPU registers, random access memory (RAM), read-only memory (ROM), and writable non-volatile memory, such as flash memory, hard drives, floppy disks, CDs, DVDs, magnetic storage devices, tape drives, device buffers, and so forth. The memory 333 stores programs and software in programming memory 334 and associated data (e.g., configuration data, settings, user options or preferences, etc.) in data memory 335. The programming memory 334 contains an operating system 336, local programs 337, and a basic input output system (BIOS) 338, all of which can be referred to collectively as general software 339. The operating system can include, for example, Microsoft Windows™, Apple iOS, Apple OS X, Linux, Android, and the like. The programming memory 334 also contains other programs and software 340 configured to perform various operations. The various programs and software can be configured to process the real-time data 107 of the athlete 111 (FIG. 2) and produce corresponding analytics, such as during the live session 51, as described in greater detail below. Those skilled in the art will appreciate that the components illustrated in the diagrams described above, and in each of the diagrams discussed below, may be altered in a variety of ways.

Clothing and Sensors

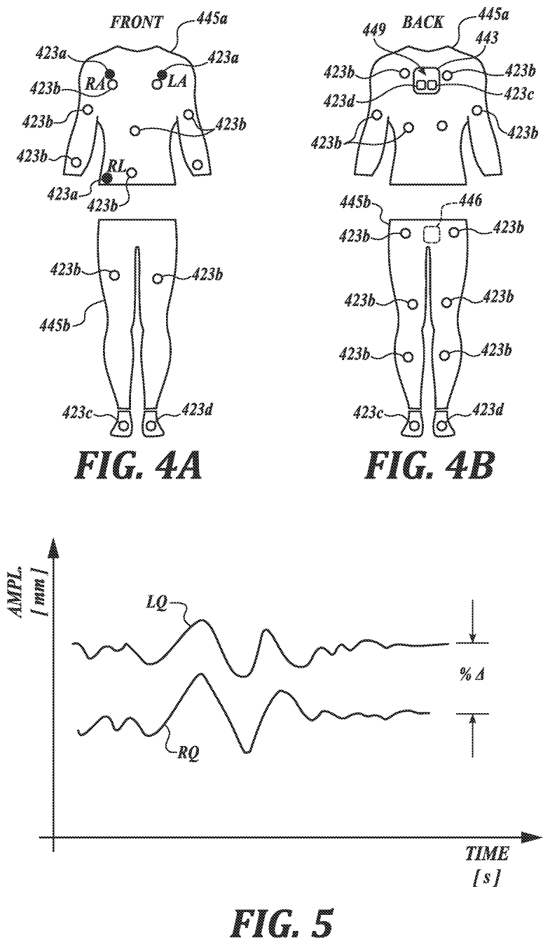

FIG. 4A and FIG. 4B are diagrams showing a measurement system in accordance with various embodiments. Referring to FIG. 5, the controller 125 can be embedded within the athlete's clothing, such as a shirt 445a and pants 445b (collectively “clothing 445”). In other embodiments, the controller 125 can be inserted into a pocket 443 in the user's clothing and/or attached using Velcro, snap, snap-fit buttons, zippers, etc. In some embodiments, the controller 125 can be removable from the clothing 445, such as for charging the controller 125. In other embodiments, the controller 125 can be permanently installed in the athlete's clothing 445.

Referring to FIG. 4A and FIG. 4B together, the controller 125 is operably coupled to muscle response sensors 423b that may be distributed over different muscle groups (e.g., pectoralis major, rectus abdominis, quadriceps femoris, biceps, triceps, deltoids, gastrocnemius, hamstring, and latissimus dorsi). The muscle response sensors 423b provide a measurement of the muscle activity during exercise. Amplitude and frequency of user's muscle response may be forwarded to the controller 125, and further to the computing devices 206 for data processing and display. A non-limiting example of the muscle response sensors 423b is an electromyography (EMG) sensor. The EMG sensors 423b can also be coupled to floating ground near the athlete's waist or hip.

In some embodiments, the clothing 445 may also be equipped with electrocardiogram (ECG) sensors 423a, orientation sensors 423c (e.g., a gyroscope), and acceleration sensors 423d (e.g., an accelerometer). Orientation sensors 423c and/or acceleration sensors 423d may be carried by the athlete's feet, for example, by being integrated and/or attached to the shoes of the athlete. The sensors 423 can be connected to the controller 125 using thin, resilient flexible wires (not shown) and/or conductive thread (not shown) woven into the clothing 445. The gauge of the wire or thread can be selected to optimize signal integrity and/or reduce electrical impedance.

The sensors 423a and 423b can include dry-surface electrodes distributed throughout the athlete's clothing 445 and positioned to make skin contact beneath the clothing along predetermined locations of the body. The fit of the clothing can be selected to be sufficiently tight to provide continuous skin contact with the individual sensors, allowing for accurate readings, while still maintaining a high-level of comfort, comparable to that of traditional compression fit shirts, pants, and similar clothing. In various embodiments, the clothing 445 can be made from compressive fit materials, such as polyester and other materials (e.g., Elastaine) for increased comfort and functionality. In some embodiments, the controller 125 and the sensors 423 can have sufficient durability and water-resistance so that they can be washed with the clothing 445 in a washing machine without causing damage. In these and other embodiments, the presence of the controller 125 and/or the sensors 423 within the clothing 445 may be virtually unnoticeable to the athlete. In one aspect of the technology, the sensors 423 can be positioned on the athlete's body without the use of tight and awkward fitting sensor bands. In the context of this application, the sensors 423 and the controller 125 are referred to as “wearable” components. In general, traditional sensor bands are typically uncomfortable for an athlete, and athletes can be reluctant to wear them.

In additional or alternate embodiments, the muscle monitor 105 (FIG. 2) can include a separate controller 446 worn on the athlete's pants 445b. The separate controller 446 can be similar to the controller 125 worn on the athlete's shirt 445a, and is connected to the individual sensors 423 located on the pants 445b. The separate controller 446 can be configured to communicate with the controller 125 and/or with the gateways 204 (FIG. 3).

Controller Communication

In operation, the controller 125 of the muscle monitor 105 is configured to process and packetize the data it receives from the sensors 423 (e.g., the muscle response sensors 423b). The controller 125 may broadcast the packetized data for detection by the gateway devices 204, which, in turn, forward the data to the muscle monitor 105 (1A) to produce analytics (e.g., frequency and amplitude of muscle activity).

Muscle Activity Indication

FIGS. 5-7 are graphs of example muscle amplitude versus time curves for two groups of muscles in accordance with various embodiments. In each graph, the horizontal axis represents time in seconds and the vertical axis represents muscle amplitude in units of displacement (e.g., mm). The illustrated graphs represent time series of the amplitude activity for particular muscle groups that was measured continuously by, for example, muscle response sensors 423b.

FIG. 5 illustrates example muscle amplitude measurements for right quad (RQ) and left quad (LQ), in accordance with various embodiments. The two muscle amplitudes retain a generally constant difference Δ, indicating a possible issue with athlete's performance (e.g., an injury or an underperformance that may be curable by better equipment of the athlete). However, the difference Δ does not increase over time, as indicated by a constant difference between the two amplitudes. In some embodiments, the system 100 may make determinations as to whether the user needs different athletic equipment and/or accessories based on the value of the difference Δ in the muscle amplitude of the RQ and LQ. In different embodiments, such threshold difference in the muscle amplitude may be normalized and expressed as:

$$Δ = \frac{LQ - RQ}{LQ + RQ} \qquad ( Eq . 1 )$$

In some embodiments, the system 100 may make determinations as to whether the user needs different athletic equipment or accessories based on the value of difference Δ in the muscle amplitude of the RQ and LQ. For example, when the value of Δ exceeds certain threshold value, the athlete may be recommended specialized athletic equipment and/or accessories. Some non-limiting sample values of the threshold Δ are 20%, 25%, 30%, 40%, 50%, or 60%.

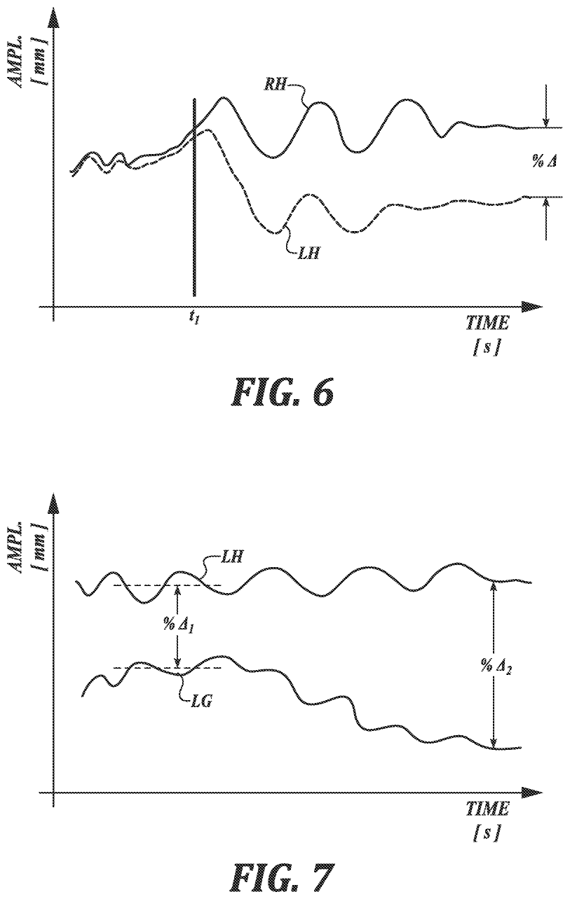

FIG. 6 illustrates example muscle amplitude measurements for right hamstring (RH, solid line) and left hamstring (LH, dash line), in accordance with various embodiments. In the beginning of the exercise and up to the time t₁, muscle amplitude for the RH and LH is generally comparable, increasing and decreasing with the intensity of exercise. In some cases, equipment and/or accessories worn or used by the athlete can influence the amplitude measurements generated by the wearable sensors. For example, some users may be sensitive to high-impact exercise and may become fatigued or experience pain that can be detected through muscle amplitude measurements. As the user becomes more fatigued in the course of the exercise, the difference in the muscle amplitude between the RH and LH becomes more pronounced. In this way, equipment that reduces the impact of the exercise will reduce the difference in the muscle amplitude. Conversely, equipment that transfers impact, such as high-stiffness tennis rackets or court shoes, for example, may amplify the difference in the signal as the athlete develops pain and favors one limb over another. In this way, an equipment recommendation could include equipment and/or accessories for use during the same exercise, such as a shoe orthotic, hamstring tape, or shock-absorbing wrap, a recommendation for a piece of equipment for a different exercise, such as a low-impact trainer (e.g., a rowing machine, an elliptical trainer, or a pilates system), or a recommendation for therapy equipment and/or accessories, such as elastic training bands, hot/cold baths, or analgesic ointments/creams. In different embodiments, such threshold difference in the muscle amplitude may be expressed as:

$$Δ = \frac{RH - LH}{RH + LH} \qquad ( Eq . 2 )$$

As explained above, different values of threshold Δ generally result in different recommendations.

FIG. 7 illustrates example muscle amplitude measurements for left hamstring (LH) and left glute (LG), in accordance with various embodiments. As described above in reference to FIG. 6, equipment recommendations may be targeted to address fatigue or injury prior to manifestation by detecting and characterizing dynamic behavior of muscle amplitude measurements. For example, as illustrated in FIG. 7, in the beginning of the exercise and up to the time t₁, muscle amplitude for the LH and LG remains within limit of Δ₁, which may be an acceptable difference based on the difference in the type of muscle. Where the athlete may be uncomfortable with the equipment, may be sensitive to impact, or may be at a level of conditioning that benefits from support equipment, as the user becomes more fatigued with the exercise, a difference in the muscle amplitude between the LH and LG becomes larger. For example, such difference may reach a value of Δ₂, indicating a zone of excessive fatigue or an increased likelihood of injury. Where a change of equipment may prevent the difference from reaching Δ₂ in future exercise or sport, a recommendation may be determined based on the value of Δ₂ and signal dynamics, such as the rate of decay in one muscle group, higher-order signal factors, that may indicate whether a change in equipment could resolve the issue. In the example illustrated in FIG. 7, the athlete is favoring the left hamstring over the left glute. In such cases, support tape may be recommended to distribute force from the LG to the LH. Similarly, to address the source of the relative weakness of LG, recommending a back-brace, where the exercise being measured follows another exercise involving weightlifting. In different embodiments, such threshold difference in the muscle amplitude may be expressed as:

$$Δ = \frac{LH - LG}{LH + LG} \qquad ( Eq . 3 )$$

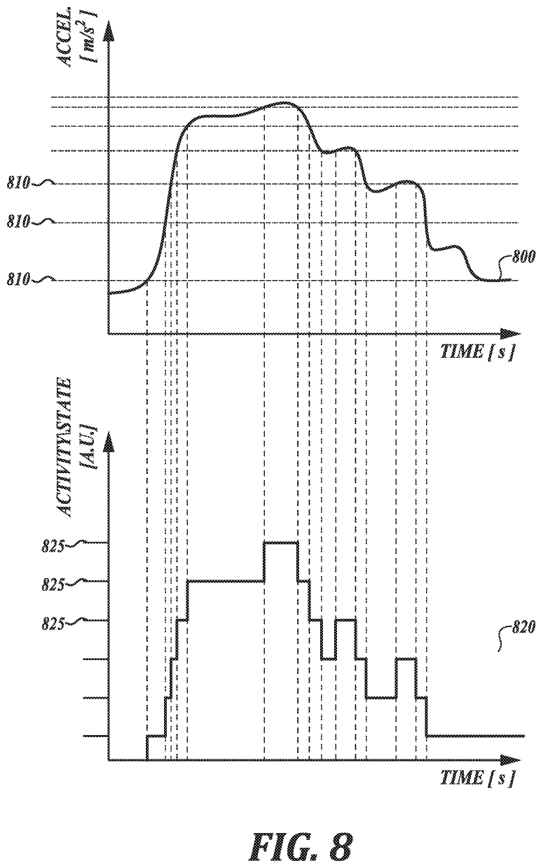

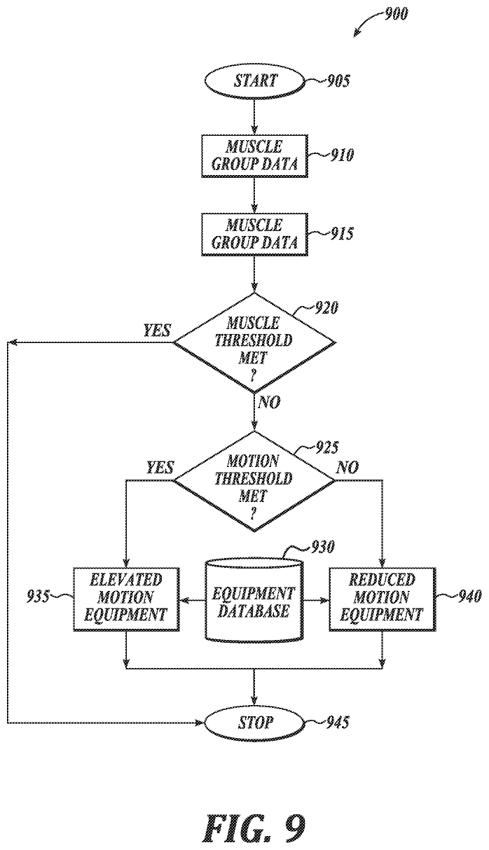

Some sample determinations of the exercise and physical therapy recommendations are described in more details with respect to FIGS. 8 and 9 below.

FIG. 8 illustrates example acceleration 800 and activity state 820 measurements, in accordance with various embodiments. As described in reference to FIGS. 1-4B, above, analytics (e.g., analytics 110 of FIG. 1) may include motion data, which may be collected during the course of a training, exercise, sport, or other activity session. Data collection may be or include continuous and/or periodic sampling of acceleration data generated by a motion sensor, such as an accelerometer, gyroscope, inertial measurement unit, or the like (e.g., accelerometer 423d of FIGS. 4A-4B). In this way, the muscle activity data, as described in more detail in reference to FIGS. 5-7, may be supplemented by motion data as part of generating an equipment recommendation.

In some embodiments, motion data may be collected by a wearable sensor borne by an athlete as part of a wearable sensor platform, as described in more detail in reference to FIGS. 4A-4B. In some embodiments, the wearable sensor platform may incorporate the accelerometer, orientation sensor, or the like, in a specific article of clothing and/or footwear, such that location-specific motion/orientation data may be collected. For example, an accelerometer and an orientation sensor may be carried in or on a shoe of an athlete, or may be worn on the foot or ankle of the athlete. In this way, an analytics system (e.g., computing device 301 of FIG. 3) may collect one or more motion signals reflecting the amplitude of motion and/or acceleration, as well as a position/orientation of the athlete's foot. With such data, the activity information may be determined, such as an activity state, equipment compatibility or fitness, whether the athlete is over-pronating or under-pronating, as well as differentiating different activity characteristics in relation to the motion signal.

In an illustrative example, the acceleration 800 amplitude signal may be implemented as part of the analytics to differentiate potentially harmful or injurious exertion at one level of motion from a generally safe exertion at another level of motion. In an illustrative example, a reinforced knee brace may be well suited for high-intensity, low-motion activity, such as squat-lifting, while a flexible sleeve may be recommended for high-motion activity, such as cardio-exercise or sprinting. In another example, where motion data indicate that repetitive stress injury may occur to the foot, ankle, or spine of the athlete, a foot orthotic or other orthopedic equipment may be recommended. Such nuances may be revealed by analyzing muscle activity data in relation to motion data.

As illustrated in FIG. 8, one or more motion levels 810 may be defined for the acceleration 800 signal. While the motion levels 810 are shown as discrete levels of acceleration, in some embodiments, the motion level is a continuous function of acceleration that is an adaptive parameter that takes into account the acceleration values preceding it. For example, in some embodiments, the motion level may be a derived value that is determined through application of one or more rules-based models or heuristics, including, but not limited to dynamic control analysis, regression model analysis, or thresholding, to derive an output signal characteristic of the activity state. Similarly, implementations of machine learning models may be trained to determine activity states using training sets derived from data collected from athletes, as described in more detail in reference to FIG. 2.

In an illustrative example, the activity state 320 may be determined in a manner analogous to a proportional-integrative-derivative signal processing techniques (PID) transfer function in t-space, where an error value e(t) is calculated as a function of time, as a measure of error between one or more motion threshold values and the acceleration 800 signal. For example, the motion level 810 measurement may be defined as a value u(t), defined as:

$$u ( t ) = K ( e ( t ) + \frac{1}{{T}_{i}} {\int }_{0}^{t} e ( {t}^{′} ) {dt}^{′} + {T}_{d} \frac{d e ( t )}{dt} ) \qquad ( Eq . 4 )$$

where K is a proportionality factor, T is a time-scale parameter over which the respective integrative “i” and derivative “d” parameters act, and e(t) is the error function, determined, for example, by comparing the acceleration 800 to a threshold value. In this way, the motion levels 810 may be predetermined or may be dynamically determined in relation to the acceleration signal and may be applied to the analytics used to process muscle signals. While the definition above for u(t) includes three terms, a simpler equation may be used that is proportional (“P”) to the error term or may use other combinations. For example, a P, a P-I, a P-D, or an I-D transfer function may be used. In an illustrative example, the motion level 810 may be a linear proportion of the acceleration 800.

In some embodiments, the motion levels 810 (u(t)) may be discretized into one of a number of activity states 825, each corresponding to a respective range of the amplitude of the acceleration 800 signal. Each activity state 825 may in turn correspond to a compensation factor that may be used by the system when developing analytics. As illustrated in FIG. 8, the activity state 825 may be constant until the acceleration 800 signal crosses a motion level 810 threshold corresponding to an activity state 825 transition.

In some cases, the activity states 825 may be defined in reference to the athlete's past performance data. The acceleration 800 signal may be tracked in a longitudinal manner over time, for multiple training sessions, exercise routines, sporting events, or the like, and may be used to define a normalization factor in reference to which the activity state 825 can be defined. For example, the activity state 825 may be normalized in reference to a pre-determined maximum value of the acceleration 800 signal. In this way, approaches including or similar to linear differentiation may be applied to analyze the input data including the acceleration 800 signal as well as muscle data, described in reference to FIGS. 5-7. Similarly, regression analysis may be applied as an approach to normalize and analyze the muscle and/or motion data, from which to make recommendations based on muscle anomalies relative to previous sessions. Advantageously, such an approach can improve training for endurance type exercise or activity and can also improve equipment recommendation, for example, by recommending professional-grade equipment when the athlete demonstrates a motion level 810 exceeding the normalization factor, equivalent to an activity state 825 greater than unity (e.g., larger than one).

Some sample determinations of the equipment recommendations are described in more detail with respect to FIG. 9.

FIG. 9 is an example flow 900 for algorithmic equipment recommendations, in accordance with various embodiments. In the illustrated embodiment, a comparison is made between groups of muscles to establish whether muscle activity is present between the groups of muscles exceeding an activity threshold in relation to a motion threshold, that in turn influences an equipment recommendation. In some embodiments, the operations of the flow 900 may include a subset of the operations illustrated, or may include additional operations that are not illustrated in the flowchart. The individual operations of the example flow 900 may be implemented by the systems described in reference to FIGS. 1-4. As such, the operations are described as part of a method implemented by a computer system. In this way, the example flow 900 may be stored as computer-executable instructions on a non-transitory computer-readable memory that, when executed by one or more processors of the computer system, may implement the operations of the flow illustrated in FIG. 9. It is understood that other systems and methods are contemplated, of which FIG. 9 describes but one example.

The method starts in block 905. In block 910, certain muscle groups are selected for observation. Some examples of such muscle groups are right quad (RQ) and left quad (LQ), right hamstring (RH) and left hamstring (LH), etc.

In block 915, motion data is collected using one or more motion sensors borne by the athlete. As described in more detail in reference to FIG. 4 and FIG. 8, the motion data may be or include, but is not limited to, the accelerometer data, orientation data, or other motion data describing position, orientation, and motion of an athlete or one or more body parts of the athlete (e.g., a foot). In some cases, the motion data collected at block 915 may be processed to provide an activity state (e.g., activity state 825 of FIG. 8) by which the muscle group data may be modified. For example, the activity state may correspond to a compensation factor applied to the muscle group data, such that a difference signal or an asymmetry signal may be amplified or damped in relation to the motion signal. In an illustrative example, asymmetric exertion may be an intended aspect of some activities, such as body-weight exercise or yoga, that are undertaken at low motion levels. In this way, a high-magnitude difference signal may be adjusted by a compensation factor smaller than one, such as about 0.1, 0.2, 0.3, 0.4, 0.5, 0.6, 0.7, 0.8, 0.9, or interpolations thereof, to reduce the influence of the muscle group data on equipment recommendations. Where analytics (e.g., analytics 110) may otherwise indicate that injury or fatigue is likely at the current conditions, and might indicate that protective equipment is suggested, a low activity state in relation to the muscle group data may permit the system to recommend professional-grade equipment that does not include protective elements with one or more accessories to target specific injury risk. Conversely, if the activity state is relatively high, the magnitude of the muscle group data signal may be amplified by the compensation factor, such that an equipment recommendation may be modified from stabilizing equipment that limits mobility to less cumbersome equipment. In an illustrative example, where a reinforced knee brace may be used for high-intensity, low-motion activity, such as squat-lifting, a flexible sleeve may be recommended for high-motion activity, such as cardio-exercise or sprinting. In another example, where the activity state indicates that repetitive stress injury may occur to the foot, ankle, or spine of the athlete, a foot orthotic or other orthopedic equipment may be recommended.

In block 920, a determination is made as to whether a muscle threshold (e.g., Δ, Δ₁, Δ₂) is met, that is, whether a difference between the measured groups of muscles is below a symmetry threshold or if the muscle group data otherwise indicates injury or fatigue are predicted. A nonlimiting example of such determination is provided in, for example, Equation 1. In the case of the first symmetry threshold, if the first symmetry threshold is met, the assumption is that the athlete is not fatigued or injured, and method may end in block 945. In the case of the second symmetry threshold, a determination is made as to whether a second symmetry threshold (e.g., Δ₂) is met, that is, whether a difference between the measured groups of muscles has reached the second symmetry threshold. In some embodiments, the second symmetry threshold indicates a condition that is more severe than the one related to the first symmetry threshold. A nonlimiting example of such determination is provided in, for example, FIG. 7, indicating that a particular problem (fatigue or injury) deteriorated further with time.

If the muscle threshold is not met, that is, a difference between the muscle amplitude of the two groups of muscles exceeds certain threshold, the system may compare the activity to a motion threshold at block 925. In block 925, a determination is made with respect to the motion data collected at block 915, which may include, but is not limited to, comparison of the motion level and/or activity state to a pre-determined motion threshold. For example, the activity state may be an integer value between one and ten, and a motion threshold may be set at an activity state value of three, such that an activity state above three is associated with fast motion and an activity state below three is associated with slow motion. In such cases where the motion threshold is met, meaning that motion data meets or exceeds the motion threshold, the system may recommend equipment for elevated motion at block 935. Other algorithms may be used in different embodiments. In different embodiments, the algorithms may be based on artificial intelligence or machine learning, as described in reference to FIG. 8, above. Such equipment recommendations may be drawn from a database 930 that includes mappings of equipment with motion and muscle data for the athlete and/or aggregated data for groups of athletes collected from prior monitored activity.

In contrast, not meeting the motion threshold causes the method to proceed to block 940 where a reduced motion equipment recommendation may be provided to the athlete. The equipment may be recommended based on data available in a database 930. In different embodiments, the database 930 may be maintained as two or more databases, for example, as part of a distributed network. The method ends in block 995.

While various advantages associated with some embodiments of the disclosure have been described above, other embodiments may also exhibit such advantages, and not all embodiments need necessarily exhibit such advantages to fall within the scope of the embodiments contemplated. For example, while various embodiments are described in the context of an athlete (e.g., a professional or collegiate athlete), in some embodiments users of the system can include novice or intermediate users, such as users, trainers, and coaches associated with a high school sports team, an athletic center, a professional gym, physical therapist, etc. Accordingly, the disclosure can encompass other embodiments not expressly shown or described herein. In the context of this disclosure, the words “approximately” or “about” indicate a difference of +/−5% of the stated value.

It is to be understood that the methods and systems described herein are not limited to specific methods, specific components, or to particular implementations. It is also to be understood that the terminology used herein is for the purpose of describing embodiments and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

What is claimed is:

1. A method for providing an equipment or accessory recommendation to an athlete, the method comprising:
monitoring a first amplitude of a first muscle of the athlete by a first wearable muscle response sensor carried by the athlete;
monitoring a second amplitude of a second muscle of the athlete by a second wearable muscle response sensor carried by the athlete;
determining a difference between the first amplitude and the second amplitude;
comparing the difference to a predetermined amplitude threshold; and
based on the comparing, providing an equipment recommendation to the athlete.

2. The method of claim 1, wherein the method further comprises:
monitoring a third amplitude of a motion signal generated by a wearable motion sensor carried by the athlete,
determining the difference between the first amplitude and the second amplitude in relation to the third amplitude, comprising:
monitoring an activity state of the athlete based on the third amplitude of the motion signal; and
applying a compensation factor to the difference or to the predetermined amplitude threshold in accordance with the activity state.

3. The method of claim 2, wherein:
the activity state is defined as the third amplitude normalized in reference to a pre-determined maximum value of the third amplitude for the athlete.

4. The method of claim 2, wherein:
the activity state is defined as a plurality of compensation factors each corresponding to a respective range of a plurality of ranges of the third amplitude; and
the compensation factor is defined as a compensation factor of the plurality of compensation factors in accordance with the third amplitude.

5. The method of claim 2, wherein the wearable motion sensor is an accelerometer that is disposed in or on a shoe worn by the athlete.

6. The method of claim 2, wherein the wearable motion sensor is an accelerometer that is worn on an ankle or a foot of the athlete.

7. The method of claim 1, wherein the equipment recommendation includes a recommendation for a foot orthotic.

8. The method of claim 1, wherein the equipment recommendation includes a recommendation for a shoe type or model.

9. The method of claim 1, wherein the first muscle is a right quad (RQ) and the second muscle is a left quad (LQ), and wherein the predetermined amplitude threshold is expressed as:
$$Δ = \frac{RQ - LQ}{RQ + LQ} .$$

10. The method of claim 9, wherein the first wearable muscle response sensor is a wearable electromyography (EMG) sensor configured for monitoring the RQ of the athlete, and the second wearable muscle response sensor is a wearable EMG sensor is configured for monitoring the LQ of the athlete.

11. The method of claim 1, wherein the predetermined amplitude threshold is 20%, 25%, 30%, 40%, 50%, or 60%.

12. The method of claim 1, wherein the first muscle is a left quad (LQ) and the second muscle is a left glute (LG), and wherein the predetermined amplitude threshold is expressed as:
$$Δ = \frac{LQ - LG}{LQ + LG} .$$

13. The method of claim 12, wherein the first wearable muscle response sensor is a wearable electromyography (EMG) sensor configured for monitoring the LG of the athlete, and the second wearable muscle response sensor is a wearable EMG sensor is configured for monitoring the LQ of the athlete.

14. A system for providing an equipment recommendation to an athlete, comprising:
a first wearable muscle response sensor configured for monitoring a first amplitude of a first muscle of the athlete;
a second wearable muscle response sensor configured for monitoring a second amplitude of a second muscle of the athlete;
a wearable motion sensor configured for monitoring a third amplitude of a motion signal generated in response to motion of the athlete;
a muscle activity tracker configured for receiving data from the first and second wearable muscle response sensors and the motion sensor and configured for determining a difference between the first amplitude and the second amplitude in relation to the third amplitude; and
at least one database storing recommendations for equipment or accessories corresponding to the determined difference between the first amplitude and the second amplitude in relation to the third amplitude.

15. The system of claim 14, wherein the system comprises one or more processors and non-transitory memory storing instructions that, when executed by the one or more processors, cause the one or more processors to generate:
a first recommendation of the recommendations in accordance with the difference failing to satisfy a predetermined amplitude threshold; and
a second recommendation of the recommendations in accordance with the difference satisfying the predetermined amplitude threshold.

16. The system of claim 15, wherein determining the difference between the first amplitude and the second amplitude in relation to the third amplitude comprises:
monitoring an activity state of the athlete using the third amplitude of the motion signal; and
applying a compensation factor to the difference or to the predetermined amplitude threshold in accordance with the activity state.

17. The system of claim 16, wherein:
the activity state is defined as the third amplitude normalized in reference to a pre-determined maximum value of the third amplitude for the athlete.

18. The system of claim 16, wherein:
the activity state is defined as a plurality of compensation factors each corresponding to a respective range of a plurality of ranges of the third amplitude; and
the compensation factor is defined as a compensation factor of the plurality of compensation factors in accordance with the third amplitude.

19. The system of claim 14, wherein the equipment recommendation includes a recommendation for a foot orthotic.

20. The system of claim 14, wherein the equipment recommendation includes a recommendation for a shoe type or model.
